(19) United States
(12) Patent Application Publication
(10) Pub. No.: US 2026/0337345 A1
(43) Pub. Date: Oct. 29, 2026

(54) PARTICULATE FILLED THERMOPLASTIC POLYMER

(71) Applicants: The University of Queensland, St. Lucia (AU); Queensland University of Technology, Brisbane (AU)

(72) Inventors: Cedryck Vaquette, Annerley (AU); Saso Ivanovski, Holland Park West (AU); Abdalla Ali, Giza (EG); Dietmar W. Hutmacher, Bellbowrie (AU)

(21) Appl. No.: 19/099,172

(22) PCT Filed: Jul. 28, 2023
(86) PCT No.: PCT/AU23/50705

§ 371 Date: Jan. 28, 2025

(30) Foreign Application Priority Data
Jul. 28, 2022 (AU) 2022902115

(51) Int. Cl. C08J 3/20; B29B 9/16; B29C 64/10; B29K 67/00; B29K 509/00; C08J 7/04; C08J 7/12; C08K 3/32; C09D 1/00

(52) U.S. Cl. CPC C08J 3/203; B29B 9/16; B29C 64/10; C08J 7/04; C08J 7/12; C08K 3/32; C09D 1/00; B29K 2067/00; B29K 2509/00; C08J 2367/04; C08K 2003/325

(57) ABSTRACT

The present invention describes a method of distributing one or more of noble metal and inorganic particles throughout a thermoplastic polymer matrix, the method comprising: providing thermoplastic polymer particles having a nucleated shell of noble metal or inorganic material, or a combination of said thermoplastic polymer particles; and melt processing the thermoplastic polymer particles; wherein melt processing of the thermoplastic polymer particles causes the nucleated shell to fragment into particles which become distributed throughout the thermoplastic polymer matrix.

### FIELD OF THE INVENTION

The present invention relates in general to particulate filled thermoplastic polymer. In particular, the invention relates to a method of distributing particulate material within a thermoplastic polymer matrix.

### BACKGROUND OF THE INVENTION

Particulate filled polymers are used extensively across a diverse range of applications. Such polymers are commonly referred to in the art as polymer composites.

The type of particulate material and polymer used varies extensively and is typically dictated by the intended application of the polymer composite.

The role and function of the particulate material can also vary extensively, but it is invariably used to impart a property to the polymer composite that cannot be achieved using the polymer alone. For example, the particulate material may function to: reduce the cost of the polymer, modify the mechanical and/or physical properties of the polymer, or impart a property of the particular material (e.g., conductivity, flame retardancy, or bioactivity) to the polymer.

The two most common ways of distributing particulate material throughout a thermoplastic polymer matrix is via solvent or melt processing techniques.

A solvent-based technique will generally involve dissolving the polymer in a solvent and mixing the particulate material into the resulting solution. The solvent is then removed leaving the particulate material distributed throughout the polymer matrix. However, the particulate material is often prone to sedimentation and/or agglomeration during removal of the solvent, the adverse effect of which can lead to non-uniform distribution of the particulate material throughout the polymer matrix, which in turn can cause embrittlement of the polymer due to the poorly distributed particulate material functioning as significant interfacial stress concentrators. Furthermore, suitable organic solvents for dissolving many commercial polymers are relatively toxic and the act of dissolving the polymer and removal of the solvent are both time-consuming and energy intensive.

Melt processing techniques can advantageously avoid the need for using solvent. However, uniformly distributing particulate material within a viscous polymer melt is problematic in its own right. For example, it is common for the particulate material to be in the form of micro- or nanoparticles, have a low bulk density, and/or possibly be opposite in polarity (i.e., hydrophobic or hydrophilic) relative to the polymer into which they are to be distributed. Such factors can make combining and efficiently melt processing the particulate material with the thermoplastic polymer particularly challenging.

Techniques to improve compatibility and mixing of particulate material with polymer during melt processing have been developed. For example, the surface properties of the particulate material can be adjusted using surface modifiers and high-shear melt processing equipment can be employed to promote intensive mixing between the polymer and the particulate material. However, the use of surface modifiers introduces a further processing step and potentially undesirable surface modifying agents into the polymer matrix, while the application of high-shear to polymer systems can adversely degrade the polymer matrix and/or the particulate material itself.

Important to the incorporation of particulate material into a polymer matrix is the substantially uniform distribution of that particulate material throughout the polymer matrix. Irrespective of the technique employed to incorporate and distribute the particulate material throughout a polymer matrix, the ability to promote a substantially uniform distribution of the particulate material throughout the polymer matrix becomes increasingly difficult as the size of the particulate material being used decreases. For example, uniformly distributing micro- or nanoparticles throughout a polymer matrix continues to present challenges to this day.

Accordingly, there remains an opportunity to develop methodology that promotes effective and efficient distribution of particulate material throughout a thermoplastic polymer matrix.

### SUMMARY OF THE INVENTION

The present invention provides a method of distributing one or more of noble metal and inorganic particles throughout a thermoplastic polymer matrix, the method comprising:
providing thermoplastic polymer particles having a nucleated shell of noble metal or inorganic material, or a combination of said thermoplastic polymer particles; and melt processing the thermoplastic polymer particles;
wherein melt processing of the thermoplastic polymer particles causes the nucleated shell to fragment into particles which become distributed throughout the thermoplastic polymer matrix. It has now surprisingly been found that upon being melt processed thermoplastic polymer particles having a nucleated shell of noble metal or inorganic material give rise to excellent distribution of noble metal and/or inorganic particles throughout the thermoplastic polymer matrix. The method provides a simple, yet highly efficient means for achieving a substantially uniform distribution of such particles throughout the polymer matrix.

Unlike conventional melt processing techniques that typically require a step of combining together as separate components the particulate material to be distributed and polymer, the method in accordance with the invention makes use of polymer that carries with it a shell precursor to the particulate material, where the particulate material that is to be distributed throughout the polymer matrix is formed in situ from the shell precursor during melt processing. Accordingly, the method in accordance with the invention avoids a step of melt processing as separate components the particulate material to be distributed and polymer. That in turn advantageously overcomes a number of problems associated with conventional melt processing techniques for incorporating particular material throughout a polymer matrix. For example, as the particles to be distributed in the polymer matrix are derived from a nucleated shell that is carried by and fixed to the polymer itself, the method in accordance with the invention advantageously minimises or avoids problems associated with any adverse mismatch in compatibility and/or bulk density between the polymer and particles.

The method of the present invention also advantageously enables one or more of noble metal and inorganic particles to be effectively and efficiently distributed throughout a thermoplastic polymer matrix without the use of solvent. The product produced in accordance with the invention may therefore be substantially free of solvent. Providing for such products that are substantially free of solvent has numerous benefits where the products are to be used in applications where the toxic nature of such solvents is detrimental.

Without wishing to be limited by theory, it is believed that while the nucleated shell associated with the polymer particles is quite robust, upon being subjected to melt processing the polymer particles melt and the nucleated shell becomes quite fragile as it is no longer being supported by an underlying solid mass. During melt processing the shell simply fragments into small particles that are inherently well-placed to become uniformly distributed throughout the polymer matrix. By being a nucleated shell, it will be appreciated it has been formed through nucleation and growth so as to coat the polymer particles. The resulting solid nucleated shell has been found to be particularly well-suited to fragmentation/disintegration during melt processing, thereby enhancing the distribution efficiency of the so-formed particles throughout the polymer matrix. The nucleated shell may partially or fully coat (i.e. encapsulate) the polymer particles.

In one embodiment, the nucleated shell encapsulates the thermoplastic polymer particles. In that case, the method of the invention may be said to provide thermoplastic polymer particles encapsulated by a nucleated shell of noble metal or inorganic material, or a combination of said thermoplastic polymer particles.

The method in accordance with the invention has been found to be particularly well-suited for use in three-dimensional (3D) polymer printing applications.

In one embodiment, the melt processing is performed as part of a three-dimensional printing process.

Further aspects and/or embodiments of the invention are described in more detail below.

### BRIEF DESCRIPTION OF THE DRAWINGS

Embodiments of the invention will now be described with reference to the following non-limiting drawings in which:

FIG. 1 illustrates calcium phosphate (CaP) shell formation on polycaprolactone (PCL) particles/pellets. A-C) Sigma 45 kDa PCL pellet coated with CaP; D-F) Polysciences Inc 50 kDa PCL, coated with CaP; G) PCL surface with simulated body fluid (SBF) ions in proximity, prior to shell formation; H) Initial surface precipitation of primarily calcium ions and limited phosphate groups, resulting in a net positive charge on the surface; I) nucleation of primarily phosphate molecules on the surface, attracted by the charge, resulting in the establishment of a mature bone-like apatite CaP surface. Scale bar for A) and D) is 1 mm, B) and E) is 1 μm, and C) and F) is 500 nm;

FIG. 2 illustrates a 3D printing process of CaP coated PCL. A) Pure PCL melt; B) SBF-PCL melt, with visible flocculates throughout the material. C) Schematic representation of the SBF-PCL processing, resulting in a nanocomposite polymer. D, E) Plain PCL after printing; F, G) PCL with ~4% CaP loading; H, I) PCL with ~6% CaP loading, inset panel showing higher magnification, arrows indicate individual CaP nanoparticles (NPs) release during sample preparation in liquid nitrogen. Scale bar for D, F, H) is 100 μm, E, G, I) is 500 nm, and inset panel in I) is 100 nm;

FIG. 3 illustrates physical characterization of PCL 3D printed scaffolds. A) Schematic illustration of elongational flow and its ability to break up particle agglomeration. B) A schematic illustration of the difference between dispersion and distribution in particle systems. C) Rheological data illustrating shear thinning properties of PCL, SBF-PCL, and HAp-PCL. The legend details the group classifications. D) TGA plot of PCL pellets, indicating the percentage of CaP loading within the 3D printed filaments over various immersion times in SBF. E) The same analysis but for PCL particles. The printed specimens for TGA were randomly selected from each group. The bottom right hand side of each curve presents the residual weight percentage related to inorganic nanoparticles loaded in PCL printed scaffolds. Inset tables show the residual weight of PCL only and PCL/CaP nanocomposite printed scaffolds, n=3;

FIG. 4 illustrates mechanical properties of PCL blends. A) Representative typical selected stress-strain plots of PCL, 7D SBF-PCL, 14D SBF-PCL, and HAp-PCL. B) Tensile elastic modulus, C) Yield stress, D) Tensile strength, and E) Strain at break for the four experimental groups. Stars indicate statistical significance against plain PCL unless indicated with lines, p<0.05. The mechanical performance of the PCL-CaP nanocomposite was enhanced compared to the plain PCL or a solution-mixed PCL/HAp composite. The nanoparticle incorporation increased the mechanical properties by homogeneously stiffening the matrix. The breaking elongation of the nanocomposites was decreased compared to plain PCL. However, efficient load transfer was found between the CaP nanofillers and the PCL matrix, resulting in improved elastic behaviour over the HAp nanocomposite. This was attributed to the agglomeration of the HAp nanoparticles, which lower the effectiveness of the nanofiller through matrix inhomogeneities and stress concentrations. Improved load transfer can only be achieved when a nanofiller is dispersed at the molecular level in the polymer matrix, as observed with the SBF-PCL groups;

FIG. 5 illustrates in vitro cell culture results of osteoblasts (alveolar bone derived-cells) seeded on 3D printed PCL-only and SBF-PCL scaffolds. a) Alamar blue assay illustrated the metabolic activity of the cells b) Picogreen assay (DNA quantification) quantified cell proliferation. c) ALP quantification. d) Absorption intensity of mineral ions released from the scaffolds after culture for 21 days;

FIG. 6 illustrates the 3D-printing approach for vertical bone augmentation within an extraskeletal ovine model. A) Timeline of the experimental approach involving a two staged strategy; bone formation followed with surgical re-entry and screw placement within formed bone B) Schematic showing the utilization of polytetrafluoroethylene (PTFE) membrane to create a dome for guided bone regeneration over the PCL-based scaffolds. The final panel shows a scanning electron microscopy imaging of the 3D-printed scaffold via fused deposition modelling (FDM); and

FIG. 7 illustrates the volume of bone formation as measured per micro-CT at 8 weeks post PCL, CaP-PCL and bone-chip-PCL (BC-PCL) scaffold placement within an extraskeletal ovine model.

### DETAILED DESCRIPTION OF THE INVENTION

The present invention provides a method of distributing one or more of noble metal and inorganic particles throughout a thermoplastic polymer matrix.

By “distributing” the particles throughout the thermoplastic polymer matrix is meant particles are spatially separated and located within a polymer matrix so as to form a polymer composite material.

The method in accordance with the invention advantageously enables the particles to be substantially uniformly distributed throughout the thermoplastic polymer matrix.

The thermoplastic polymer matrix may have distributed therethrough one or both of the noble metal and inorganic particles.

As will be discussed in more detail below, the noble metal and inorganic particles are respectively derived from a nucleated shell of noble metal or inorganic material.

There is no particular limitation on the size of the noble metal or inorganic particles, but they will often be microparticles or nanoparticles.

In one embodiment, the noble metal and inorganic particles are microparticles or nanoparticles. The noble metal and/or inorganic particles may be present as a combination of microparticles and nanoparticles.

Reference herein to a microparticle is intended to mean particles of matter having at least one dimension less than 1000 microns (μm). The term “microparticle” is of course also intended to embrace particles of matter in which all dimensions are less than 1000 μm.

While the term “microparticle” is broad enough to embrace the term “nanoparticle”, the term “nanoparticle” is often used to define a smaller subset of microparticle.

As used herein, the term “nanoparticle” is intended to mean a particle of matter having at least one dimension less than 100 nanometres (nm). A nanoparticle therefore also includes particles of matter having all dimensions less than 100 nm.

For avoidance of any confusion in using both the terms “microparticle” and “nanoparticle” herein, a microparticle will be confined to mean particles of matter having at least one (or all) dimension(s) from 100 nm to less than 1000 μm, whereas a nanoparticle will remain as being defined as a particle of matter having at least one (or all) dimension(s) of less than 100 nm.

The present invention makes use of thermoplastic polymer. Those skilled in the art will appreciate that in contrast with a thermoset polymer, a thermoplastic polymer softens upon being heated and can be molded into a desired shape. Thermoplastic polymers therefore are amenable to melt processing and can typically also be dissolved in a solvent.

Provided a nucleated shell of noble metal or inorganic material can be formed on particles of the thermoplastic polymer, there is no particular limitation on the type of thermoplastic polymer that may be used in accordance with the invention.

Examples of suitable thermoplastic polymers include, but are not limited to, polyester, polyamide, polyolefin, polyurethane, polystyrene, polycarbonate, and polyacrylate.

In one embodiment, the thermoplastic polymer is selected from polyester, polyamide, polyolefin, polyurethane, polystyrene, polycarbonate, polyacrylate.

In a further embodiment, the thermoplastic polymer is a biodegradable thermoplastic polymer. By the thermoplastic polymer being “biodegradable” is meant the polymer is capable of undergoing a significant degradation of is molecular structure under specific environmental conditions resulting in a loss of properties as measured by standard test methods appropriate to the polymer and the application in a period of time that determines its classification. Biodegradable polymers undergo degradation through the action of naturally-occurring micro-organisms such as bacteria, fungi, and algae (see also ASTM D883-20a).

In a further embodiment, the thermoplastic polymer is a polyester or polyamide.

In another embodiment, the thermoplastic polymer is a biodegradable polyester or polyamide. Examples of suitable polyesters include, but are not limited to, polycaprolactone, polylactic acid, polygylcolic acid, polyhydroxy butyrate, polyethylene succinate, polybutylene adipate terephthalate, polyhydroxy butyrate valerate, polybutylene succinate, polybutylene adipate, cellulose acetate butyrate, cellulose acetate propionate.

In one embodiment, the thermoplastic polymer is a polyester selected from polycaprolactone, polylactic acid, polygylcolic acid, polyhydroxy butyrate, polyethylene succinate, polybutylene adipate terephthalate, polyhydroxy butyrate valerate, polybutylene succinate, polybutylene adipate, cellulose acetate butyrate, cellulose acetate propionate.

Examples of suitable polyamides include, but are not limited to, polyamide 6, polyamide 12, polyamide 6/6, polyamide 6/9, polyamide 6/10, polyamide 6/12, polyamide 4/6, and polyamide 12/12.

In one embodiment, the thermoplastic polymer is a polyamide selected from polyamide 6, polyamide 12, polyamide 6/6, polyamide 6/9, polyamide 6/10, polyamide 6/12, polyamide 4/6, and polyamide 12/12.

In a further embodiment, the thermoplastic polymer is polycaprolactone.

In another embodiment, the thermoplastic polymer is selected from low-density polyethylene (LDPE), high-density polyethylene (HDPE), polypropylene (PP), polyvinyl chloride (PVC), thermoplastic polyurethanes (TPU), polychlorotrifluoroethylene (PCTFE), and polyacrylonitrile (PAN).

Noble metal and/or inorganic particles are distributed throughout the thermoplastic polymer matrix. Reference herein to the thermoplastic polymer “matrix” is intended to mean the polymer composition per se that is made up of an entanglement of the polymer chains.

Distribution of the noble metal and/or inorganic particles throughout the thermoplastic polymer matrix can be readily evaluated through imaging techniques such as scanning electron microscopy (SEM).

The method according to the invention comprises providing thermoplastic polymer particles having a nucleated shell of noble metal or inorganic material, or a combination of said thermoplastic polymer particles.

Reference herein to thermoplastic polymer “particles” is intended to mean thermoplastic polymer as described herein provided in the physical form of particulate matter. Provided the thermoplastic polymer particles can accommodate the nucleated shell, there is no particular limitation on the size or shape of those polymer particles.

For example, the thermoplastic polymer particles can have a spherical/bead or pellet-like shape. The thermoplastic polymer particles may have a largest average dimension (e.g. diameter in the case of a spherical-like shape) ranging from about 10 μm to about 5 mm.

The thermoplastic polymer particles may be produced from the base thermoplastic polymer by any suitable means. For example, the particles may be produced by conventional granulation techniques such as pelletising, ball milling and cryo-milling.

The thermoplastic polymer particles have a nucleated shell of noble metal or inorganic material. Reference herein to a “nucleated shell” is intended to mean a shell of the specified material that has been formed through a nucleation and growth process. Accordingly, the nucleated shell in accordance with the present invention represents a shell of noble metal or inorganic material that has been formed at the surface of the thermoplastic polymer particles through nucleation and growth. The nucleation may give rise to growth of one or both of crystalline and amorphous noble metal or inorganic material. Following nucleation of the noble metal or inorganic material at the polymer particle surface, growth occurs and continues across the surface of the polymer particle so as to form the nucleated shell.

In one embodiment, the nucleated shell comprises crystalline noble metal or inorganic material. In another embodiment, the nucleated shell comprises amorphous noble metal or inorganic material.

In a further embodiment, the nucleated shell comprises a combination of crystalline and amorphous noble metal or inorganic material.

The nucleated shell may partially or fully coat (i.e. encapsulate) the polymer particles.

In one embodiment, the nucleated shell encapsulates the thermoplastic polymer particles. In that case, the method of the invention may be said to provide thermoplastic polymer particles encapsulated by a nucleated shell of noble metal or inorganic material, or a combination of said thermoplastic polymer particles.

The nucleated shell can be readily identified as such by virtue of the shell being made up of crystalline and/or amorphous matter adhered to the surface of the polymer particle. That crystalline or amorphous matter can be readily determined to have been formed through a nucleation and growth process at the polymer surface through, for example, microscopy techniques such as scanning electron microscopy or energy dispersive X-ray spectroscopy or Transmission electron microscopy or Fourier-transform infrared spectroscopy.

There is no particular limitation on the thickness of the nucleated shell, which will typically be determined by the methodology used to form the shell. Generally, the nucleated shell will have a thickness ranging from about 100 nm to about 50 μm.

The nucleated shell may be made of noble metal. As used herein, the expression “noble metal” is intended to be a reference to ruthenium, rhodium, palladium, silver, osmium, iridium, platinum, and gold. The nucleated shell may comprise two or more noble metals.

The nucleated shell may be made of inorganic material. Provided the inorganic material can form a nucleated shell on the thermoplastic polymer particles, there is no particular limitation on the type of inorganic material that can be used. All inorganic materials that can nucleate on the surface of the polymer particles and undergo growth to coat the particles are suitable for use in accordance with the invention. Examples of such inorganic material includes, but are not limited to, calcium phosphate, titanium dioxide, zinc oxide, magnesium hydroxide, silicon dioxide, cadmium selenide, cadmium telluride, zirconium oxide, aluminium oxide, iron oxide, calcium oxide, manganese oxide, lead sulfide, cadmium sulfide, and copper oxide.

In one embodiment, the nucleated shell comprises inorganic material selected from calcium phosphate, titanium dioxide, zinc oxide, magnesium hydroxide, silicon dioxide, cadmium selenide, cadmium telluride, zirconium oxide, aluminium oxide, iron oxide, calcium oxide, manganese oxide, lead sulfide, cadmium sulfide, copper oxide and combinations thereof.

The nucleated shell may be made of two or more different inorganic materials.

The thermoplastic polymer particles used in accordance with the invention may comprise a mixture of thermoplastic polymer particles having different nucleated shells as described herein.

The thermoplastic polymer particles having the nucleated shell may be prepared by any suitable means. By virtue of having a nucleated shell, the process of preparing the polymer particles used in accordance with the invention will inherently involve a step of nucleation of the noble metal or inorganic material at the surface of the base polymer particles, followed by a growth phase so as to form the nucleated shell.

In one embodiment, the thermoplastic polymer particles having a nucleated shell are prepared by a process comprising (i) exposing a polymer surface of the thermoplastic polymer particles to a nucleating species to produce a nucleation site on that surface, and (ii) exposing that nucleated site to a source of the noble metal or inorganic material, which in turn promotes growth of the noble metal or inorganic material and forms the nucleated shell of that material.

The nucleated shell may form partially or fully around the polymer particle. Where growth occurs over the entire surface of the polymer particles the nucleated shell can be described as encapsulating the polymer particles.

In one embodiment, growth of the noble metal or inorganic material provides for a nucleated shell that encapsulates the polymer particles.

One approach to prepare the thermoplastic polymer particles having a nucleated shell includes exposing a polymer surface of the thermoplastic polymer particles to an aqueous solution comprising ions of the inorganic material that is to form the nucleated shell. Ions in the solution can deposit on the surface of the polymer particles so as to form nucleation sites from which growth of the inorganic material occurs that propagates so as to form the nucleated shell.

In one embodiment, the thermoplastic polymer particles having a nucleated shell are prepared by a process comprising (i) exposing a polymer surface of the thermoplastic polymer particles to a nucleating species to produce a nucleation site on that surface, and (ii) exposing that nucleated site to a source of the noble metal or inorganic material, which in turn promotes growth of the noble metal or inorganic material and forms the nucleated shell of that material.

In one embodiment, the so formed nucleated shell comprises calcium phosphate and the aqueous solution comprises calcium and phosphate ions.

In addition to the ions of the inorganic material that is to form the nucleated shell, the aqueous solution exposed to the polymer surface of the thermoplastic polymer particles may comprise one or more additives. For example, those additives could be used to facilitate growth of the inorganic material and may include, for example, one or more other inorganic materials and buffer for regulating pH.

In one embodiment, the aqueous solution comprising ions of the inorganic material is a simulated body fluid (SBF).

The amount of time taken to form the nucleated shell will vary depending upon the material from which the shell is to be made, the technique used to form the shell, the required degree of coverage of the shell on the thermoplastic polymer particles, and the desired thickness of the shell. The nucleated shell can be formed over the period of a few hours or up to several days.

Where the nucleated shell is formed using an aqueous solution of ions that form the nucleated shell of inorganic material, the ions of the inorganic material will generally be present in the solution at a concentration ranging from about 0.01 to about 1 mmol/dm³.

A typical composition for a SBF suitable for use in accordance with the invention includes that shown in the table directly below:

Ion composition of a typical simulated body fluid (mmol/dm³)

| Na⁺ | K⁺ | Ca²⁺ | Mg²⁺ | Cl⁻ | HCO₃⁻ | HPO₄²⁻ | SO₄²⁻ |
|---|---|---|---|---|---|---|---|
| 141.7 | 5.8 | 1.3 | 0.9 | 147.7 | 4.2 | 0.78 | 0.4 |

Depending upon the composition of the nucleated shell and thermoplastic polymer on which the shell is to be formed, the polymer surface of the thermoplastic polymer particles on to which the nucleated shell forms may benefit from undergoing a surface treatment to assist with nucleation and shell formation.

In one embodiment, the polymer surface of the thermoplastic polymer particles on which the nucleated shell forms is subjected to a surface treatment prior to formation of the nucleated shell. Generally, such treatment of the polymer surface will be used to create surface functional groups that assist with the nucleation step in the nucleated shell formation. For example, where the nucleated shell is made of inorganic material, that inorganic material will typically be made up of positive and negative ions. The process of forming such a nucleated shell might involve exposing a polymer surface of the thermoplastic polymer particles to an aqueous solution comprising positive and negative ions of the inorganic material. In that case, it may be desirable to modify the surface of polymer exposed to the aqueous solution by, for example, increasing the amount of negative ions at the surface. Having negative ions at the polymer surface can promote attraction of positive ions of the inorganic material from the aqueous solution leading to a net positive charge on the surface of the polymer. That net positive charge can then in turn promote association of negative ions of the inorganic material present in the aqueous solution and consequently give rise to nucleation sites from which growth of the inorganic material can propagate so as to form the nucleated shell.

Depending upon the nature of the thermoplastic polymer and nucleated shell to be employed, those skilled in the art would be able to readily evaluate if surface treatment of the polymer surface on to which a nucleated shell is to be deposited might assist with formation of the nucleated shell. Suitable surface treatments include, but are not limited to, oxygen plasma treatment, alkaline treatment, acid treatment and aminolysis.

In one embodiment, the thermoplastic polymer particles comprise polyester or polyamide and a surface on to which the nucleated shell is to be formed has undergone an alkaline surface treatment.

Alkaline treatment may be performed by any suitable means, for example, the polymer surface may be exposed to an aqueous alkaline solution, for example, an aqueous solution comprising an alkali metal hydroxide (e.g., sodium hydroxide or potassium hydroxide).

In one embodiment, the surface of polymer on to which the nucleated shell is to be formed undergoes alkaline surface treatment by being exposed to an aqueous solution comprising an alkali metal hydroxide.

Surface treatment of the thermoplastic polymer particles may, for example, increase the density of functional groups such as hydroxyl, carboxyl and amine groups, which in turn can assist with formation of the nucleated shell.

The thermoplastic polymer particles having a nucleated shell are subjected to melt processing in accordance with the method of the invention.

Melt processing in accordance with the present invention is intended to mean the thermoplastic polymer particles are heated to a temperature at or above the polymers' melting point such that the polymer forms a molten mass and flows. As those skilled in the art will appreciate, melt processing will typically also involve subjecting the polymer to some form of shear force so as to at least promote flow of the molten polymer and generally also melt mixing of the polymer.

Melt processing of the thermoplastic polymer particles in accordance with the invention causes the nucleated shell to fragment into particles that become distributed throughout the thermoplastic polymer matrix. Provided the nucleated shell fragments into particles there is no particular limitation on the equipment that may be used to melt process the thermoplastic polymer particles. Conventional melt processing equipment can advantageously be used in accordance with the invention and include, but are not limited to, melt extrusion equipment, three dimensional melt printing equipment, melt electrospinning equipment and melt injection equipment.

As those skilled in the art will appreciate, through at least inducing melt flow and also through melt mixing, melt processing equipment subjects the polymer melt to shear forces. On being subjected to melt processing, polymer of the thermoplastic polymer particles becomes molten and through shear forces invoked at least through polymer flow and/or mechanical mixing the nucleated shell readily fragments into particles and inherently becomes distributed throughout the thermoplastic polymer matrix.

In one embodiment, melt processing is conducted using a technique selected from melt extrusion, three dimensional melt printing, melt electrospinning and melt injection equipment.

The polymer melt produced in accordance with the method of the invention can advantageously be formed into any desired shaped product, with the resulting thermoplastic polymer product of course having noble metal and/or inorganic particles distributed throughout the polymer matrix. The nucleated shell per se that presents on the thermoplastic polymer particles is inherently relatively fragile and therefore readily fragments into particles during melt processing. The size of the particles formed through fragmentation of the nucleated shell will vary depending upon the thickness of the shell, the nature of the shell, and also the amount of shear applied to the polymer melt during melt processing. Generally, an increased amount of shear applied to the polymer melt during melt processing will reduce the particle size derived from the fragmented shell.

In one embodiment, the nucleated shell fragments into microparticles.

In a further embodiment, the nucleated shell fragments into nanoparticles.

Melt processing of the thermoplastic polymer particles facilitates distribution of the noble metal or inorganic particles throughout the polymer matrix. The method accordance with the invention advantageously enables the noble metal or inorganic particles to be uniformly or home ingeniously distributed throughout the polymer matrix.

The amount of noble metal and/or inorganic particles that are ultimately distributed throughout the thermoplastic polymer matrix can be readily adjusted by varying the number of thermoplastic polymer particles used that have the nucleated shell and/or by varying the thickness of the nucleated shell together with the degree of shell encapsulation (i.e., whether or not the thermoplastic polymer particles are partially or fully encapsulated by the nucleated shell).

The method of the present invention is particularly well-suited to producing biodegradable thermoplastic polymer scaffolds having a polymer matrix in which is distributed inorganic and/or noble metal particles.

In one embodiment, the thermoplastic polymer is biodegradable and polymer melt produced by the melt processing is formed into a scaffold for tissue engineering.

Polymer products such as tissue engineering scaffolds produced in accordance with the invention advantageously exhibit a number of improved properties.

For example, polymer products produced in accordance with the invention can present a polymer matrix having a substantially uniform distribution of noble metal and/or inorganic particles. That excellent distribution of the particulate material throughout the polymer matrix imparts numerous improved mechanical properties to the product. For example, the polymer can exhibit improved tensile elastic modulus, yield stress and/or tensile strength, relative to the base thermoplastic polymer absent the particulate material.

The method in accordance with the present invention has been found to be particularly well-suited to using three dimensional melt printing to produce biodegradable tissue engineering scaffolds that exhibit excellent mechanical properties and can inherently possess within their polymer matrix inorganic materials such as calcium phosphate, which is particularly advantageous in tissue engineering applications.

In one embodiment, the thermoplastic polymer is biodegradable polyester or polyamides and the thermoplastic polymer particles have a calcium phosphate nucleated shell.

In a further embodiment, the thermoplastic polymer particles having a calcium phosphate nucleated shell are melt processed using three-dimensional melt printing and formed into a scaffold for tissue engineering.

The three-dimensional melt printing may be performed by extrusion of the thermoplastic polymer particles through nozzles having a diameter that ranges from about 0.1 to about 5 mm.

The present invention will hereinafter be described with reference to non-limiting examples.

### EXAMPLES

uNucleation and Growth of CaP Minerals on Polymers Substrates

Poly(caprolactone) was used in two different forms from different sources, pellets (~3.532 mm size) with an number-average molecular weight (M₁) of 45 kDa (Sigma Aldrich) and irregular particles (<600 μm average particle size) with M₁ 50 kDa (Polysciences, Inc.). The material density of polycaprolactone is 1.145 g/cm³ at 25 °C. The melting point is around 60 °C and melt index is 1.8 g·10 min⁻¹ (80 °C, 44 psi).

Both PCL pellets and irregular particles were chemically treated with 3 M NaOH for 3 h at 37 °C. Subsequently, the treated PCL substrates were subjected to alternate calcium and phosphate-ion dipping treatment. The PCL substrates were initially placed into 150 mM CaCl₂) solution for 30 s, then washed with Milli-Q water for 5 s. Then, the samples were dipped in 150 mM Na₂HPO₄ solution for 30 s and washed again. This process was repeated six times before incubation in simulated body fluid (SBF), at 37 °C for 1, 5, 7 and 14 days to investigate the mineralization behavior on both PCL pellets and particles. After removal from the SBF, the PCL pellets/irregular particles was gently washed with Milli-Q water, then dried in air at room temperature for 3 days before further processing.

### Fabrication of 3D Nanocomposite Scaffold Using BioScaffolder

The coated PCL pellets/irregular particles with CaP minerals were used to additively manufacture a 3D structure layer by layer using a 3D bioprinter (GeSiM BioScaffolder 3.1, GeSiM mbH, GroGerkmannsdorf, Germany and 3D Bioplotter EnvisionTEC) with TECDIA Arque 500, 400 and 300 μm inner diameter precision dispensing needles (TECDIA Inc., Campbell, CA, USA). The printing process was performed at 95 °C, with an extrusion air pressure of 550 kPa, and an extruder speed of 4 mm·s⁻¹. Scaffolds composed of six layers per construct were produced (height approximately 2.2 mm), 1.0 mm filament spacing, and a 900 angle of rotation between subsequent layers.

For comparison, a conventional solvent blending method was also used, where 4.3 wt. % of synthesized hydroxyapatite nanoparticles was added into a PCL solution (10 wt. % of PCL pellets in chloroform). Hydroxyapatite powders were synthesized by a wet chemical precipitation method. Briefly, 1 M Ca(NO₃)₂·4H₂O aqueous solution was prepared and subsequently 0.6 M (NH₄)₂HPO₄ solution was added dropwise under strong stirring. After stirring the solution for 3 h at room temperature (RT), the slurry was heated to 90 °C for 2 h under continuous stirring. After filtering the milky solution, the precipitate was washed several times with a mixture of distilled water and 100% ethanol (volume ratio 1:1) until the pH was neutralized. The synthesized powder was dried under vacuum for 24 h at 100 °C and thereafter combined to the PCL-chloroform solution. The solvent was evaporated and the composite was manually sectioned in pellets and use for 3D-printing

### Scanning Electron Microscopy of Coated PCL Substrates and Printed 3D Scaffolds

The coated PCL pellets/irregular particle substrates with CaP minerals and the printed nanocomposites were imaged using a scanning electron microscope (SEM, Jeol 7001F, Japan) operated with an acceleration voltage of 10 kV equipped with energy dispersive spectroscopy (EDS) for elemental analysis. Sample cross sections were produced by immersing the samples in liquid N₂ for 10 min, then rapidly removing them from the N₂ and immediately manually fracturing them across the fibers. Before imaging, the samples were coated with a 3 nm platinum layer using a compact coating unit (CCU-010, Safematic).

### Alizarin Red Staining

Alizarin red staining was performed to assess CaP deposition on PCL powder substrates. Specimens were covered in 500 μL of 40 mM alizarin red S dye solution at pH 4.1 for 30 min. The dye was eliminated by rinsing multiple times in pure Milli-Q water until clear water remained. Scaffolds were air-dried at RT for 30 min prior to imaging. To visualize the formation of CaP, 2-D images of PCL-only and PCL coated with CaP were obtained.

### Example 1

### Part (a) Shell Formation

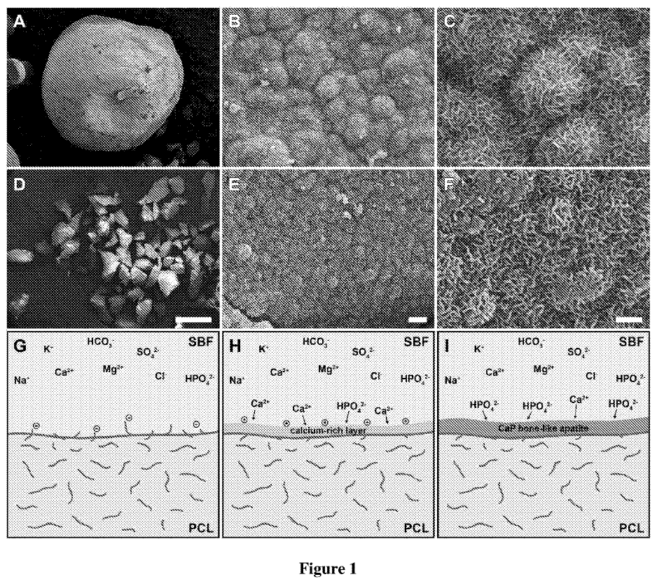

This approach was implemented in the present work by first chemically etching poly(ε-caprolactone) (PCL) pellets in an alkaline medium. This surface modification cleaved superficial PCL chains, forming a reactive layer rich in hydroxyl and carboxyl groups. The pellets were then incubated in SBF to enable the in situ nucleation and growth of calcium phosphate crystals via the alternate adsorption of apatite precursors, calcium (Ca²⁺) cations and phosphate (HPO₄²⁻) anions (FIG. 1). The incubation resulted in the formation of a stable uniform coating over 5 and 7 days. After 14 days, however, a thick but brittle and unstable shell was obtained. The coating was characterized with scanning electron microscopy (SEM; FIG. 1) and energy-dispersive X-ray spectroscopy (EDXS), which demonstrated that it comprised of multiple nanoscale CaP particles, and confirmed the calcium and phosphorus ratio as 1.4 Ca/P, with magnesium and sodium in quantities below 1 atomic percent. The mineralized products formed apatite crystals.

### Part (b) Fabrication of the Composite Scaffold Using Melt Extrusion

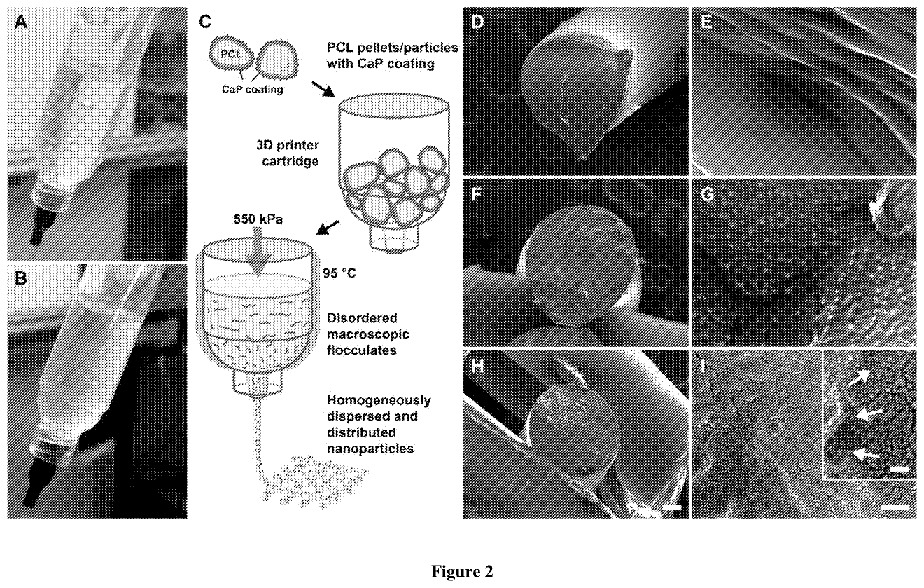

The coated pellets were placed in a 10 mL syringe dispensing chamber (Nordson Corporation, USA) and heated to a temperature of 95 °C using a GeSiM BioScaffolder 3.1 (GeSiMmbH, Radeberg, Germany). Upon the melting of the polymer, the CaP shell underwent a visible macroscopic disintegration, resulting in a poorly homogenized suspension of CaP particles in the polymer melt (FIG. 2). The CaP/PCL mixture was then 3D printed using 550 kPa pressure and a 500 μm nozzle (Tecdia Arqué stainless steel needle ARQ-S-4050; Tecdia INC, CA, USA). The resultant polymer blend contained a highly homogenous dispersion of CaP NPs throughout the PCL bulk (FIG. 2). To our knowledge, this level of reproducible and consistently homogeneous distribution and dispersion of NPs through the bulk of a thermoplastic polymer has not been demonstrated in the literature.

We propose that the specific flow conditions of the extrusion process induced the disassembling of the micro-scale CaP plates/particles into highly stable, unit-cell type nanoparticles, and homogenously dispersed them throughout the extruded filaments (FIG. 2D-I, FIG. 2A, B). This process was performed with regular, spherical PCL pellets, and irregular, rhomboid PCL particles. High magnification SEM images of printed filament cross-sections (FIG. 2G, I) were used for the quantification of the average particle size, which was performed using ImageJ software (NIH, USA). The CaP particle sizes within the printed matrix were about 46.68±5.92 nm and 17±2.3 nm for the 3D printed matrix obtained from PCL pellets and the irregular PCL particles, respectively. High resolution SEM was performed across samples produced in three independent manufacturing runs to ensure the reproducibility of the process, and to characterize the CaP particle size and distribution at filament cross-sections from a larger sample size. As a comparative reference, PCL without a CaP shell was also printed and the filament cross-section was imaged, revealing a smooth surface (FIG. 2D, E).

### Part (c) Dispersion Mechanism

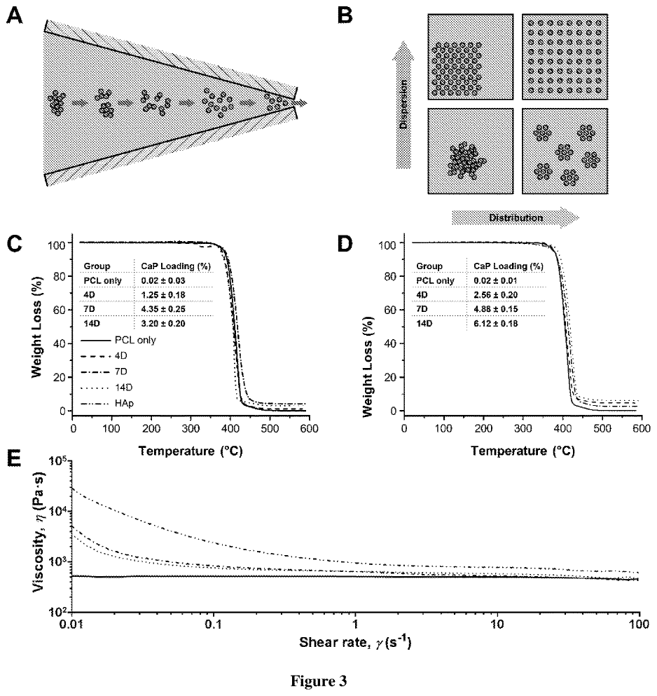

Dispersion of particles in a polymer melt is likely due to the presence of repulsive forces between the particles as they approach each other. It has been previously demonstrated that the unique combination of shear and elongational flow, as encountered in the tapering section of the cartridge and the needle, can result in the dispersion of NPs and the reduction of agglomeration (FIG. 3A). While the sequential breakdown of particles has been shown to occur under a sustained shear stress (i.e. not combined shear and elongational flow) in the context of a microfluidic emulsification process, such stress conditions are largely considered to lead to particle agglomeration. When applied in a non-Newtonian fluid and with incompressible particles, the tension in surrounding streamlines applies a net force on adjacent particles, pushing them together, and essentially resulting in the accumulation of particles aligned with the streamline, in the direction of the flow. This is an analogous phenomenon to what is commonly observed with the melt processing of large and mechanically robust particles, whereby the high shear rate at the wall results in a high streamline tension and subsequently the movement of particles towards the center of the pipe cross-section. An important point to note regarding this phenomenon, however, is that particle alignment is dependent on many factors, including the matrix viscosity, the shear rate, and the concentration of the particles, with both steric and hydrodynamic interactions manifesting at high shear rates and particle concentrations.

Elongational flow entails a stretching of the material itself, with the fluid deformation affecting the particles. This is generally present in thixotropic (shear thinning) materials, where the phenomenon may be exploited to promote particle alignment perpendicular to the axis of flow, rather than parallel, as is the case with shear flow. Elongational flow has also been demonstrated to impart improved morphology and higher entanglement within a polymer, compared with the use of an exclusively shear flow process.

In the present study, the specific conditions of the combined shear and elongational flow in the cartridge and throughout the needle resulted in the manifestation of a number of the above phenomena, with the rheological properties of the polymer synergizing with the highly brittle and thin CaP shell. These effects not only induced the dispersion of agglomeration and the formation of distinct nanoparticles, but the homogeneous distribution of these particles throughout the bulk of the polymer (FIG. 3B).

### Part (d) Nozzle Size

To further explore the effect of flow conditions on nanoparticle dispersion and distribution, the effect of nozzle diameter was investigated using a range of high precision nozzles of different diameters. No remarkable differences were observed between the different nozzles, with the CaP NPs homogenously dispersed in each tested case. This confirmed that the flow conditions within the syringe and not inside the nozzle were responsible for producing the observed phenomenon. Additional evidence supporting the phenomenon occurring inside the syringe is that significant CaP disintegration at the entrance of the nozzle would likely result in macro particle formation, and ultimately nozzle blockage during the printing process.

### Part (e) Elemental Mapping

The printing of the CaP-coated pellets resulted in a highly homogenous dispersion of CaP NPs of a predicted elemental composition. The CaP NPs were evenly distributed with a similar inter-particle distance, suggesting that the CaP shell was extensively broken down and physically rearranged throughout the process, presumably due to the flow conditions of the system. No particle aggregation was found in any of the independent specimens that were imaged. We further demonstrated that our method was effective when using PCL polymer obtained from different manufacturers, with the dispersion and distribution of nanoparticles in this PCL matrix also being highly homogenous.

### Example 2 Physiochemical Analysis

### X-Ray Diffraction (XRD) of 3D Printed Filaments

XRD patterns were recorded by a Siemens D5000 diffractometer equipped with an X-ray generator (λ=0.15406 nm) between 5 and 700 with increments of 0.02°. 3D printed sheets were prepared for this purpose.

Elemental Chemical Analysis of CaP Nanoparticles Nucleated from SBF onto PCL Pellets

To chemically analyse the nucleated and growth of CaP shell nanocrystals, the PCL pellets were sonicated for 10 min at room temperature to peel off the CaP shell layer from the polymer substrate. The samples were analyzed at 670.783 nm wavelength using a Varian (brand, manufactured Melbourne Australia) Vista Pro (model) radial ICP-OES instrument. Standards from 0-5 mg/L CaP were prepared from Fluka, TraceCERT 1000 mg/L stock standard. Results reported are the average of three second integrations.

### Thermogravimetric Analysis (TGA)

To measure the mineralized CaP nanoparticle loading within printed composite scaffolds, TGA was performed under nitrogen at a heating rate of 10 K·min⁻¹ from room temperature up to 600 °C by using a Hi-Res TGA 2950 from TA Instruments. About 20-25 mg of sample was used in each run.

### Differential Scanning Calorimeter (DSC)

Thermal analysis of the as-received, untreated and thermally treated PCL polymer was analysed using a differential scanning calorimeter (DSC, Q20V24.9) equipped with a liquid nitrogen cooling accessory. The samples were placed in aluminium pans under a nitrogen atmosphere, heated from −80 to 110 °C, cooled to RT, and then heated to 110 °C and maintained for 1 min to ensure the complete melting of the PCL crystals. The samples were subsequently quenched to −80 °C at the same rate of 10 °C·min⁻¹ and then heated again from −80 °C to 110 °C at the same heating rate. The data was collected from the second cycle. The glass transition temperatures (T_{g}) were determined from the inflection point of the heat flow curve. The thermograms were recorded at a rate of 10 °C·min⁻¹. The melting points and the heat of fusion (the melting heat) were determined to calculate the polymer crystallinity. First and second independent cycles of heating and cooling runs were initiated in which the first cycle was able to eliminate the polymer heat history. With a reference point of 136 J·g⁻¹ for 100% crystalline PCL, the degree of crystallinity X_{c} % of the polymer scaffold was determined. Of note, the composite scaffolds containing CaP particles had a lower contribution from PCL than the plain PCL scaffolds. Hence, the heat of fusion of the composite was calculated thus:

$$\frac{heat of fusion of plain PCL \times 100}{PCL concentration in the composite matrix}$$

### Part (a) XRD and Elemental Analysis

The crystallographic nature of the CaP NPs was investigated by XRD, demonstrating that CaP was present in the 3D printed CaP-PCL nanocomposite. The amorphous nature that is characteristic of CaP was illustrated through the relatively broad X-ray diffraction peaks, in contrast with the sharp peaks often present with highly-crystalline hydroxyapatite (HAp). This amorphous form of CaP is thought to arise from the incorporation of impurities within the matrix, including carbonate, sodium and magnesium ions (as confirmed by ICPOES analysis, Table 3).

TABLE 3
ICPOAS chemical analysis of nucleated and grown CaP
shell onto PCL pellets after peeling off under 10 min sonication

| Na | K | Ca | Mg | Cl | CO₃ | P | SO₄ |
|---|---|---|---|---|---|---|---|
| mg/l | mg/l | mg/l | mg/l | mg/l | mg/l | mg/l | mg/l |
| 70.4 | 7.42 | 4250 | 121 | 4.1 | 51.4 | 1283 | 76 |

### Part (b) Thermal Analysis

Thermogravimetric analysis (TGA) was utilized to quantify the loading of the CaP NPs into the printed filaments, and demonstrated that the loading ranged from 2-6%, depending on the SBF immersion time and the geometrical structure of the PCL substrate (FIG. 3C, D). Interestingly, the CaP loading was partly dependent on the surface area to volume ratio of the polymer substrate. The irregular particles are considerably smaller (>600 m) than the pellets (>4 mm), and hence the same CaP coating thickness could result in the incorporation of a lot more CaP into the polymer matrix. Indeed, delamination of the CaP nucleated shell was observed when spherical polymer pellets were immersed for a 14-day time point, while an irregular-shaped PCL powder displayed a stable coating at this time. This may be attributed to the nature of CaP nucleation, where predominantly flat surfaces tend to provide a more stable area for aggregation and crystal growth, while curved surfaces may result in the accumulation of stress concentrations in parts of the coating and cause cracking once certain thresholds are reached.

Part (c) Rheometry Properties of PCL-Only and PCL Substrates Coated with CaP Minerals

Plain PCL and PCL pellets coated with CaP, incubated in SBF for 7 and 14 days, were processed in an Anton Parr M302 Rheometer laboratory internal mixer at 60 rpm for 20 min, with the chamber wall kept at 95 °C. Before applying the test, the samples were heated to 95 °C and kept inside the chamber for 30 min to reach equilibrium. Viscosity and shear stress as functions of shear rate were plotted for PCL and the composites over a strain rate range of 0.01 to 100 s⁻¹.

The rheological properties of PCL and the CaP nanoparticle-containing PCL were assessed to determine any effects of the particles on the flow characteristics of the material (FIG. 3E). The particles were found to primarily induce a thixotropic behavior on the composite, in contrast with the largely Newtonian character of PCL. The CaP-PCL was found to possess a viscosity (η) in excess of 5×10³ Pa·s at low shear rates (~0.01 s⁻¹), which was around one order of magnitude higher than plain PCL (~500 Pa·s), reaching a comparable viscosity by a shear rate of 10 s⁻¹. A solvent mixed hydroxyapatite (HAp)-PCL control was also included in the assessment, and demonstrated a similar thixotropic characteristic. However, the viscosity of the HAp-PCL did not reach that of PCL at high shear rates, remaining ~200 Pa·s higher, and the viscosity at low shear rates was almost an order of magnitude higher than the CaP-PCL, reaching around 3×10⁴ Pa·s.

### Example 3

### Mechanical Properties

Plain PCL and SBF-PCL individual printed filaments were subjected to mechanical testing using an Instron Universal tester model: M10-14190-EN, LR5K plus, UK with a 500 N capacity load cell. Samples were vertically mounted on two mechanical gripping units of the tensile tester at their ends. The filament samples were glued onto individual paper frames to ensure a constant gauge length of 20 mm. For the 3D printed filaments (6 layers), sample dimensions with 5 mm thickness×60 mm length (20 mm gauge length)×2.2 mm height were used. Testing was conducted with the grips moving at a rate of 3 mm·min⁻¹. The load was applied until the specimens experienced complete failure. The individual and 3D fused filament thicknesses were precisely measured using a digital micrometer with a precision of 1 μm before running the test. The tensile modulus was calculated as the slope of the initial linear portion of the stress-strain curve. The data acquisition rate was set to 20.0 Hz. All of the experiments were conducted at room temperature. Five to seven samples were tested for each batch at room temperature. The data presented are expressed as mean±standard deviation.

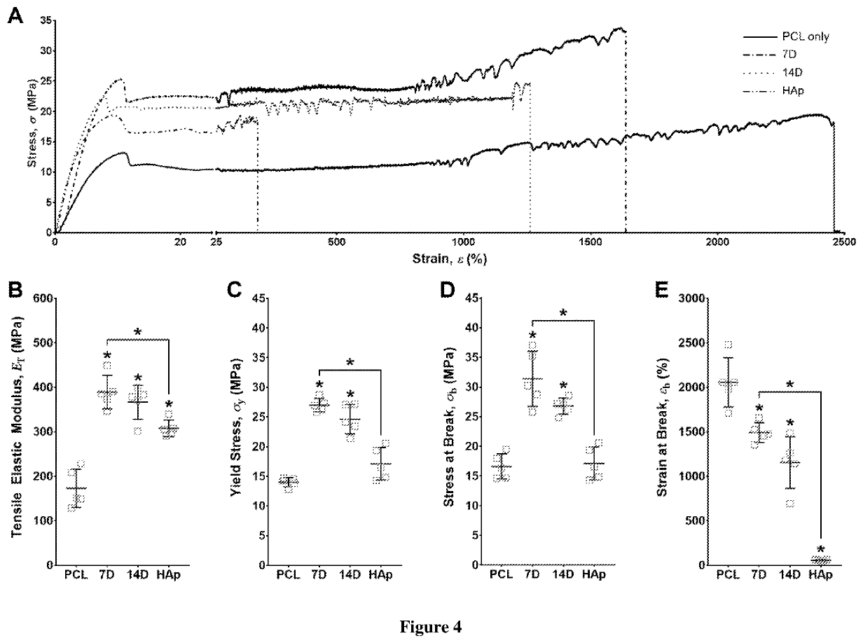

Mechanical testing was conducted on both CaP-PCL and plain PCL, and significant (~2-fold) increases over the plain PCL control were observed in the tensile elastic modulus, yield stress, and tensile strength of the composite (FIG. 4). Furthermore, it was found that the mechanical properties of the nanocomposite were consistent between blends formed with two different types of PCL (i.e. two morphologies, spherical polymer pellets, 45 kDa M., Sigma Aldrich, Merck KGaA, Germany, and a polymer powder, 50 kDa MW, Polysciences, Inc., PA USA). To precisely determine the mechanical properties before and after incorporation of CaP NPs, individual filaments were tested in tension (representative stress-strain plot in FIG. 4A; also refer to FIG. 4). All CaP-PCL nanocomposites achieved very high elongation before breaking, in excess of 1250% strain. This is in contrast with a HAp-PCL conventional solvent blend, which broke around 250% strain, and plain PCL, which reached ~2500% strain at breaking (FIG. 4E). This property may be attributed to the homogeneous dispersion of the nanoparticles achieving a consistent stress distribution throughout the matrix[1-3], unlike that present within the solvent mix of HAp nanoparticles and PCL.

The tensile elastic modulus, yield stress, and tensile strength of the nanocomposites were all found to be significantly (p<0.05) higher than plain PCL (FIG. 4B-D). The samples also exhibited a similar stress-strain behavior, with a sharp increase in stress upon loading, and a gradual increase to the strength prior to fracture. The enhancement of mechanical properties largely coincided with an increase in nanoparticle loading, although the 14-day loading provided marginal and inconsistent increases above the 7-day group across both of the tested types of PCL. Scaffolds printed from the CaP-PCL composite showed a similar improvement in mechanical properties to the individual strands.

### Example 4

### In Vitro Assessment of Human Osteoblasts Cultured on 3D Printed Constructs

Osteoblast cell culture: Following institutional ethics approval, cells obtained from one patient undergoing dental surgery were used in this study. Portions of alveolar bone removed during tooth extraction were chipped into small fragments and immersed in media (DMEM high glucose, Invitrogen, CA, USA) supplemented with 10% Fetal Bovine Serum (FBS, Thermo Fisher Scientific, MA, USA) and 4% Penicillin-Streptomycin (reduced to 1% after 24 h) and incubated at 37 °C with 5% CO₂. Outgrowth of osteoblasts was observed after 5-7 days. All in vitro cell culture studies were conducted using cells at passage 5.

Cell seeding: Scaffolds were immersed in FBS for 2 h at 37 °C. Excess of FBS was removed and scaffolds were seeded with 25 000 cells in 30 μL of basal media (DMEM; 1% PS; 10% FBS). Cells were allowed to attach for 2 h and scaffolds were grown in both basal and osteogenic media (basal media supplemented with 50 μg·mL⁻¹ ascorbate-2-phosphate (AA), 0.1 μM Dexamethasone and 10 mM β-glycerophosphate). Media was changed every 2/3 days.

Cell Metabolism: Cell metabolism was assessed using the Alamar blue assay according to the manufacturer's protocol. Briefly, culture medium was replaced with medium containing 10% (v/v) Alamar blue solution (Invitrogen), and the cells were incubated at 37 °C for 4 h. Absorbance was measured at 570 and 600 nm using a plate reader (POLARstar Omega, BMG LABTECH, Germany) and the % reduction of Alamar blue was calculated in each well. Cell metabolism was analyzed on day 3, 7 and 21 (n=4).

Cell Proliferation: At 3, 7 and 21 days, the scaffolds were removed from the culture medium, washed in PBS and frozen at −80 °C until further processing. Subsequently, they were digested overnight at 56 °C in a Tris-EDTA buffered solution containing Proteinase-K (0.5 mg·mL⁻¹).

DNA quantification assay was performed with the PicoGreen dsDNA assay according to the manufacturer's instructions (P11496, Invitrogen), using a spectrofluorometer (POLARstar Omega), at an excitation wavelength of 485 nm and an emission wavelength of 520 nm.

Cell Morphology and Attachment: At selected time points (3, 7 and 21 days; n=3) cell attachment on the PCL scaffolds was assessed qualitatively by staining with 4,6-diamino-2-phenylindole (DAPI, Life Technologies, NY, USA) and Alexa Fluor 568 Phalloidin (Life Technologies, Grand Island, NY, USA). Scaffolds were rinsed in PBS then fixed for 1 h in 4% paraformaldehyde (PFA) in PBS. Cells were then incubated for 10 min in PBS containing 5 μg·mL⁻¹ DAPI and 0.8 U·mL⁻¹ Alexa Fluor 568 Phalloidin. Scaffolds were rinsed in PBS then imaged using the Leica TCS SP5 scanning laser confocal microscope (Leica Microsystems, Mannheim, Germany).

Alkaline phosphatase (ALP) activity: ALP activity, an early marker of osteogenesis, was measured to study cell activity on the scaffolds at various incubation times (days 3, 7, 21). As per the manufacturer's instructions (Thermo Fisher Scientific), lysis buffer was added to the samples, which were then incubated at 4 °C for 30 min, followed by centrifugation at 12,000 rpm for 10 min at 4 °C. Equal volumes of sample solution and Alkaline Phosphatase Yellow (pNPP) Liquid Substrate where plated in a 96-well plate, and plates were incubated for 1 h in the dark at 37 °C. The absorbance at 405 nm was measured using a microplate reader. The results were expressed as units per milligram of protein in cell lysate, where the protein was assayed by the Bradford assay (B6916, Sigma Aldrich) method, using bovine serum albumin as standard.

Alizarin red staining and quantification: For characterization of the mineralized matrix after 21 days of culture, scaffolds were fixed with 4% paraformaldehyde and stained with 1% Alizarin Red S solution in water for 30 min at room temperature. After this, samples were rinsed in excess Milli-Q water until all unbound dye was removed, and dried at RT for 30 min prior to imaging. The samples were imaged under an inverted phase-contrast microscope. For quantitative analysis, the staining was dissolved in 10% cetylpyridinium chloride (Sigma) in PBS and the absorbance was measured at 540 nm using a microplate reader.

### Data Analysis

Each experiment was performed at least three times. All quantitative data was expressed as mean±standard deviation. One way analysis of variance (ANOVA) was used to test the null hypothesis, that the group means were equal, against an alternative hypothesis, that at least two of the group means were different, at an α=0.05 significance level (SPSS 24.0, IBM Corporation, NY, USA). When the p-value resulting from the initial ANOVA model was determined to be <0.05, multiple comparisons were made between group means with Tukey's post-hoc test.

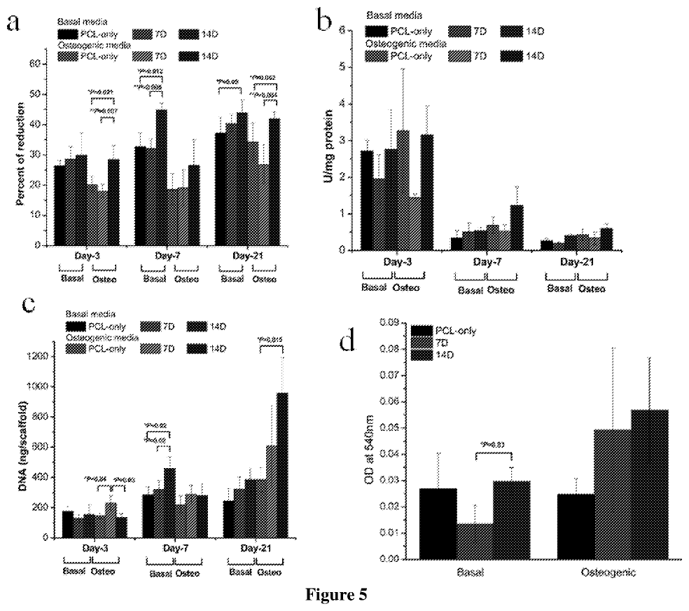

The cell biocompatibility of the CaP-PCL scaffolds were evaluated in vitro with primary human osteoblasts. The nanocomposite scaffolds supported cell attachment, migration, and proliferation, as well as the formation of a cohesive extracellular matrix throughout the entire interconnected pore architecture (FIG. 5). This further supports the utility of this approach, and illustrates the possibility of using such scaffolds in biomedical applications.

### Example 5

### Calculation of Number of Particles Per Mm for the Printed Nanocomposites Filament Matrix

The average number of particles embedded in 3D printed filament matrix was calculated as following. Briefly, for the PCL pellets coated with CaP, the coatings were considered as spherical shells with an outer radius R and inner radius r.

Before deposition of CaP minerals, 25 PCL pellets (spheres) were measured using a digital micrometer and the average diameter was calculated. The coated layer thickness of CaP minerals was measured by SEM. The average diameter of the PCL spheres was 3663±14 μm. So by taking:

$$V = \frac{4}{3} π {R}^{3}$$

And the volume of the shell of the sphere:

$$V = \frac{4}{3} π {R}^{3} - \frac{4}{3} π {r}^{3}$$

We were able to calculate the approximate volume of a thin spherical shell for each ball, which is the area of the outer sphere multiplied by the thickness t of the shell:

V≈4πR²t

When t (0.43 μm) is very small compared to R (3663 μm) (t<<R)
So, V (the volume of the coated layer)=10539597.22 μm³

V=Vₚ (volume of one CaP particle, measure from SEM cross section)×Nₚ (number of CaP particles)
Hence Nₚ=39298924286 particles=112×10⁹ (112 Billion particles)

Thus, it can be concluded that there are 112 billion particles deposited on each coated PCL sphere. Furthermore, the volume of the PCL spheres can be used to calculate that one sphere is sufficient to produce a 56.2 mm filament with a 500 μm cross-sectional diameter, as per the below equation for the volume of a cylinder.

V=πr²h

Therefore, the number of particles per 1 mm length of a printed single PCL filament are 112×10⁹/56.2=~2×10⁹, and each 1 mm of printed PCL filament possesses ~2 billion particles of CaP.

### Example 6

### In Vivo Assessment in an Extraskeletal Ovine Model

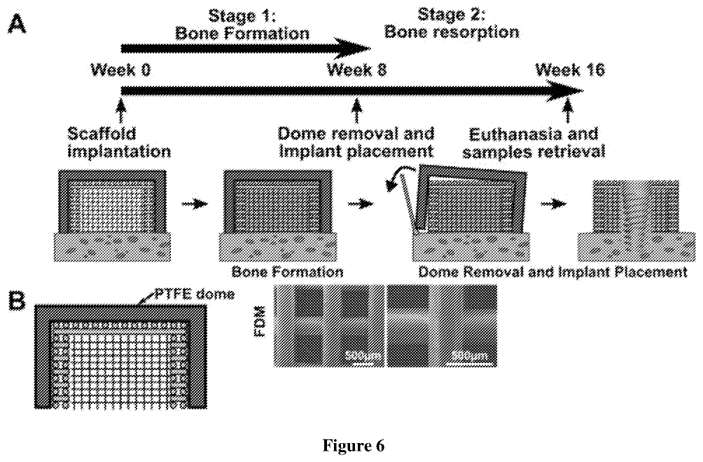

An ovine extraskeletal bone formation model was utilized where the scaffold was placed as an onlay graft over the sheep calvarial bone. A polytetrafluoroethylene dome was positioned over the scaffolds to act both as a space maintainer and as an occlusive barrier preventing soft tissue infiltration (FIG. 6). Three groups were implanted in 5 animals for 16 weeks: 1) CaP-PCL, 2) PCL, 3) PCL loaded with bone chips (BC-PCL).

Circumferential grooves (10 mm and 0.5 mm deep) were created in the calvarial bone and within each groove 8-10 perforating holes (1 mm in diameter, 2 mm in depth) were drilled through the cranial cortical plate to allow bleeding. The constructs were randomly positioned over the grooves and stabilized using titanium retention pins followed by closure of the wound.

Following a 8-week healing period, a surgical re-entry was performed and the protective polytetrafluoroethylene domes were removed. A bone biopsy (2.8 mm external diameter) was taken from one specimen of each group in order to place a commercially available dental implant (SLActive, 8 mm long 3.3 mm diameter, Straumann, Basel Switzerland). After a further 8 weeks of healing, the animals were euthanized and the samples retrieved. Micro-CT scans were utilized to determine bone volume around the implant and over the calvarial bone. Micro-CT scanning (pCT40, SCANCO Medical AG, Bruttisellen, Switzerland) was performed at a resolution of 30 μm, voltage of 70 kVP, current of 114 μA, power of 8 W, an integration time of 200 ms and a greyscale threshold of 220. Bone formation within the elevated space was quantified by manually segmenting the newly formed bone.

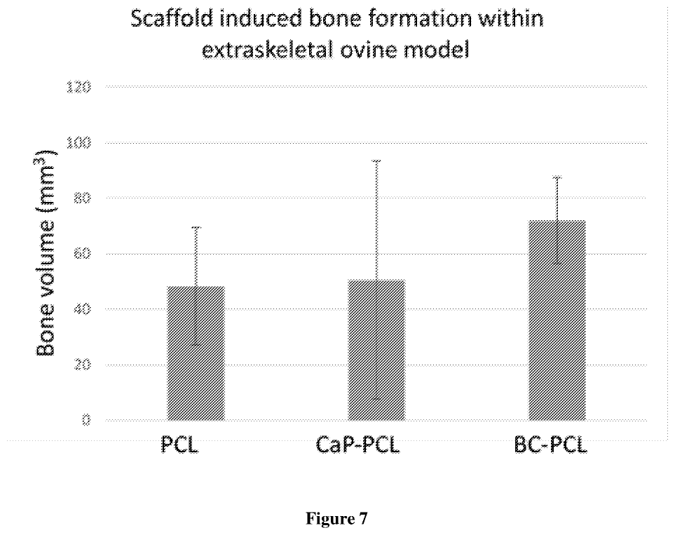

This experiment demonstrated that all groups enabled the maintenance of the bone previously formed subsequent to dental implant placement (FIG. 7). This also revealed that the CaP-PCL was well tolerated and integrated within the host tissue demonstrating biocompatibility. The CaP-PCL performed similarly to plain PCL and PCL loaded with bone chips (a clinical standard to enhance bone formation) demonstrating that bioactivity and adequate space maintenance are the determining factors for osteogenesis to occur in the context of vertical bone formation.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

1. A method of distributing one or more of noble metal and inorganic particles throughout a thermoplastic polymer matrix, the method comprising:
providing thermoplastic polymer particles having a nucleated shell of noble metal or inorganic material, or a combination of said thermoplastic polymer particles; and
melt processing the thermoplastic polymer particles;
wherein melt processing of the thermoplastic polymer particles causes the nucleated shell to fragment into particles which become distributed throughout the thermoplastic polymer matrix.

2. The method according to claim 1, wherein the nucleated shell fragments into nanoparticles.

3. The method according to claim 1, wherein the thermoplastic polymer is selected from polyester, polyamide, polyolefin, polyurethane, polystyrene, polycarbonate, and polyacrylate.

4. The method according to claim 3, wherein the thermoplastic polymer is a biodegradable polyester or polyamide.

5. The method according to claim 4, wherein the biodegradable polyester is selected from polycaprolactone, polylactic acid, polygylcolic acid, polyhydroxy butyrate, polyethylene succinate, polybutylene adipate terephthalate, polyhydroxy butyrate valerate, polybutylene succinate, polybutylene adipate, cellulose acetate butyrate, and cellulose acetate propionate.

6. The method according to claim 2, wherein the nucleated shell comprises inorganic material selected from calcium phosphate, titanium dioxide, zinc oxide, magnesium hydroxide, silicon dioxide, cadmium selenide, cadmium telluride, zirconium oxide, aluminium oxide, iron oxide, calcium oxide, manganese oxide, lead sulfide, cadmium sulfide, copper oxide and combinations thereof.

7. The method according to claim 1, wherein the nucleated shell encapsulates the thermoplastic polymer particles.

8. The method according to claim 7, wherein the thermoplastic polymer particles having a nucleated shell are prepared by a process comprising (i) exposing a polymer surface of the thermoplastic polymer particles to a nucleating species to produce a nucleation site on that surface, and (ii) exposing that nucleated site to a source of the noble metal or inorganic material, which in turn promotes growth of the noble metal or inorganic material and forms the nucleated shell of that material.

9. The method according to claim 7, wherein the thermoplastic polymer particles have an inorganic nucleated shell and are prepared by a process comprising (i) exposing a polymer surface of the thermoplastic polymer particles to an aqueous solution comprising ions of inorganic material that form a nucleation site on the polymer surface, and (ii) incubating the thermoplastic polymer particles in the aqueous solution to promote growth of inorganic material from the nucleated sites and formation of the nucleated shell of inorganic material.

10. The method according to claim 9, wherein the aqueous solution is a simulated body fluid.

11. The method according to claim 8, wherein the polymer surface of the thermoplastic polymer particles on which the nucleated shell forms is subjected to a surface treatment prior to formation of the nucleated shell.

12. The method according to claim 11, wherein the surface treatment is selected from oxygen plasma treatment, alkaline treatment, acid treatment and aminolysis.

13. The method according to claim 1, wherein the nucleated shell comprises calcium phosphate.

14. The method according to claim 1, wherein melt processing is conducted using a technique selected from melt extrusion, three dimensional melt printing, melt electrospinning and melt injection.

15. The method according to claim 1, wherein the thermoplastic polymer particles (i) have a calcium phosphate nucleated shell, (ii) are melt processed using three-dimensional melt printing, and (iii) formed into a scaffold for tissue engineering.
